(19) United States
(12) Patent Application Publication
(10) Pub. No.: US 2016/0022623 A1
(43) Pub. Date: Jan. 28, 2016

(54) COMPOSITION AND USE

(71) Applicant: THE UNIVERSITY OF NOTTINGHAM, Nottingham (GB)

(72) Inventors: Paul Greenhaff, Nottingham (GB); Francis Stephens, Nottingham (GB)

(21) Appl. No.: 14/773,292

(22) PCT Filed: Mar. 7, 2014
(86) PCT No.: PCT/GB2014/050672

§ 371 Date: Sep. 4, 2015

(30) Foreign Application Priority Data
Mar. 7, 2013 (GB) 1304112.4

(51) Int. Cl. A61K 31/205; A23L 1/305; A23L 1/29; A61K 31/70; A61K 38/02

(52) U.S. Cl. CPC A61K 31/205; A61K 31/70; A61K 38/02; A23L 1/293; A23L 1/3051; A23V 2002/00

(57) ABSTRACT

The invention provides the simultaneous, separate or sequential feeding or administration of a carnitine substance (e.g. L-carnitine) with an agent for increasing the blood/plasma insulin concentration. The agent may be protein, carbohydrate or one or more amino acids or a combination of such nutrients. The co-administration or co-feeding of the carnitine substance and the agent provide for the reduction of whole body fat mass, particularly associated with excess dietary energy intake. The disclosed co-administration may also provide for the treatment of insulin resistance, fat oxidation disorders or obesity, and for increasing energy expenditure and fat metabolism. Compositions and kits containing the carnitine substance and the agent are provided, as are methods of treatment employing the co-administration or feeding of the carnitine substance and the agent. Also described is the upregulation of several genes in response to the treatments of the invention, including ACAT1, PNPLA2, PDK2, FOXO3, TFAM and CPT1.

### TECHNICAL FIELD

The invention relates to the field of dietary supplementation and the modulation of energy metabolism during dietary supplementation. In particular, the invention relates to the field of enhancing fat metabolism and reducing body-mass gain brought about by excess dietary energy intake. The invention also relates to the reduction of obesity and insulin resistance.

### INTRODUCTION

As a substrate for carnitine palmitoyltransferase 1 (CPT1), carnitine translocates long-chain acyl groups across the otherwise impermeable inner mitochondrial membrane for subsequent β-oxidation. A series of studies from the inventors laboratory have suggested that an insulin-mediated augmentation of skeletal muscle total carnitine content can increase CPT 1 flux and mitochondrial long-chain acyl group translocation in vivo, and result in a reciprocal inhibition of carbohydrate utilisation. By way of example, the inventors have demonstrated that a 15% increase in skeletal muscle total carnitine content, achieved via intravenous L-carnitine infusion during a 6 h euglycaemic hyperinsulinaemic clamp in healthy human volunteers at rest, decreased insulin stimulated muscle pyruvate dehydrogenase complex (PDC) activation and muscle lactate accumulation by 30 and 40%, respectively, compared to control (euglycaemic hyperinsulinaemia). These findings were particularly remarkable as during hyperinsulinaemic conditions cytosolic malonyl-CoA, which is a potent inhibitor of CPT1, would likely be elevated because of increased glycolytic flux. Furthermore, the morning following the euglycaemic hyperinsulinaemic clamp in this same study, where a controlled diet was consumed prior to an overnight fast, muscle long-chain acyl-CoA and glycogen content had increased by 40 and 30%, respectively, suggesting that a carnitine mediated increase in long-chain acyl group translocation had diverted glucose uptake from oxidation towards storage. More recently the inventors demonstrated that it is possible to increase muscle total carnitine content via dietary means, for example, by ingesting 1.36 g L-carnitine in combination with a beverage containing 80 g of carbohydrate (in order to stimulate insulin mediated muscle carnitine accumulation) twice daily over a 24 week period, which resulted in a 30% increase in muscle total carnitine content compared to carbohydrate feeding alone. This manipulation of the muscle total carnitine pool resulted in an 80% increase in muscle free carnitine availability and a remarkable 50% reduction in muscle glycogen utilisation during low intensity exercise (50% of maximal oxygen consumption).

There is a need to provide dietary supplements and supplementing regimes and/or methods of treatment capable of modulating human energy metabolism. In particular there is a need for new dietary regimes capable of reducing body-mass accumulation associated with excess dietary energy intake. There is also a need for a dietary supplementation regime capable of modulating fat metabolism and reducing the accumulation of body fat mass associated with excess dietary energy intake. There is also a need for dietary supplements and regimes capable of modulating fat oxidation and a subject's sensitivity to insulin.

Such regimes would be advantageous for the safe supplementation of diet with carbohydrates, proteins and amino acids, associated with sports activities, and would provide health benefits by increasing fat metabolism e.g. for the reduction of obesity and insulin resistance.

The invention solves these and other problems by providing, in a first aspect, a carnitine substance for use in the reduction of whole-body fat mass accumulation, wherein the carnitine substance is administered or used as a dietary supplement together with an agent to increase blood/plasma insulin concentration. The agent to increase blood/plasma insulin concentration may be a carbohydrate, protein, one or more amino acids or a combination of such nutrients.

In a related aspect, the invention provides an agent for use in the reduction of whole-body fat mass accumulation, wherein the agent is administered or used as a dietary supplement together with administration of or dietary supplementation with a carnitine substance, and wherein the agent is suitable for increasing the blood/plasma insulin concentration. The agent may be a carbohydrate, protein, one or more amino acids or a combination of such nutrients.

The invention further provides a carnitine substance for use in the treatment of insulin resistance, fat oxidation disorders and/or obesity, wherein the carnitine substance is administered or used as a dietary supplement together with an agent to increase blood/plasma insulin concentration. Similarly, the invention provides an agent for use in the treatment of insulin resistance, fat oxidation disorders or obesity, wherein the agent is administered or used as a dietary supplement together with administration of, or dietary supplementation with a carnitine substance, and wherein the agent is suitable for increasing the blood/plasma insulin concentration. The agent may be a carbohydrate, protein, one or more amino acids or a combination of such nutrients.

In another aspect the invention provides a carnitine substance for use in increasing energy expenditure and fat metabolism in a subject, wherein the carnitine substance is administered or used as a dietary supplement together with an agent to increase blood/plasma insulin concentration. The agent may be a carbohydrate, protein, one or more amino acids or a combination of such nutrients.

The invention also provides an agent for use in increasing energy expenditure and fat metabolism in a subject, wherein the agent is administered or used as a dietary supplement together with administration of, or dietary supplementation with a carnitine substance, and wherein the agent is suitable for increasing the blood/plasma insulin concentration. The agent may be a carbohydrate, protein, one or more amino acids or a combination of such nutrients.

In each of the above aspects, a carnitine substance and the agent for increasing the blood/plasma insulin concentration may be administered or fed to a subject simultaneously, separately or sequentially. In some aspects the carnitine substance and carbohydrate are fed or administered to a subject in a composition comprising both. The invention therefore provides a composition comprising a carnitine substance and an agent for increasing the blood/plasma insulin concentration for use in the reduction of whole-body fat mass accumulation, the treatment of insulin resistance, fat oxidation disorders or obesity, or increasing energy expenditure and fat metabolism in a subject.

Similarly, the invention provides a kit comprising a carnitine substance and an agent for increasing the blood/plasma insulin concentration, and instructions for use in the reduction of whole-body fat mass accumulation, the treatment of insulin resistance, fat oxidation disorders or obesity, or increasing energy expenditure and fat metabolism in a subject.

The invention also provides methods for treatment using a carnitine substance and an agent for increasing the blood/plasma insulin concentration. In some aspects, the invention therefore provides a method for reducing whole-body fat mass accumulation comprising administering an agent for increasing the blood/plasma insulin concentration and a carnitine substance to an individual or supplementing the diet of an individual with an agent for increasing the blood/plasma insulin concentration and a carnitine substance.

The invention further provides a method for treating insulin resistance, fat oxidation disorders or obesity comprising administering an agent for increasing the blood/plasma insulin concentration and a carnitine substance to an individual or supplementing the diet of an individual with carbohydrate, protein, one or more amino acids or a combination of such nutrients to increase blood/plasma insulin concentration and a carnitine substance.

The invention further provides a method for increasing energy expenditure and fat metabolism in a subject, comprising administering an agent for increasing the blood/plasma insulin concentration and a carnitine substance to an individual or supplementing the diet of an individual with an agent for increasing the blood/plasma insulin concentration and a carnitine substance. The agent may be a carbohydrate, protein, one or more amino acids or a combination of such nutrients.

In some embodiments of each of the above aspects, a carnitine substance is administered or fed at a dose of about 0.5 to 4 g per day, preferably at a dose of about 1 to 3 g per day, e.g. 1.5 to 2.5 g per day. Preferably this carnitine dose is divided over two doses per day, e.g. 0.25 to 2 g twice per day. But carnitine may also be administered in one or more doses per day, e.g. one dose per day, two doses per day, or three or more doses per day. In some embodiments the carnitine dose is about 2.72 g per day, e.g. about 1.36 g twice per day.

In some embodiments of each of the above aspects, the agent for increasing the blood/plasma insulin concentration is administered or fed at a total dose of about 20 g to about 200 g per day. Preferably the agent dose is about 50 g to 150 g per day, e.g about 100 g to 125 g. The agent may be administered in one or more doses per day, e.g. one dose per day, two doses per day, or three or more doses per day. In some embodiments, the agent is administered or fed at about 160 g per day, e.g. about 80 g twice per day. The dose of the agent is preferably adequate to elevate blood/plasma insulin to about 50 mU/L or more and may be administered concurrent with carnitine ingestion.

The inventors have shown the upregulation of a range of genes upon the administration or feeding of an agent for increasing the blood/plasma insulin concentration and a carnitine substance as described herein. The invention therefore provides for each of the above aspects of the invention in which any one or more of the genes of Table 1 are upregulated. In particular, the invention provides for any of the above noted aspects, in which one or more genes involved in insulin signalling, PPAR signalling or fatty acid metabolism is upregulated by the administration or dietary supplementation with carnitine and an agent for increasing the blood/plasma insulin concentration. The agent may be a carbohydrate, protein, one or more amino acids or a combination of such nutrients

The invention further provides for any of the above noted aspects in which the expression of one or more of ACAT1, PNPLA2, PDK2, FOXO3, TFAM or CPT1 is upregulated by the administration of, or dietary supplementation with a carnitine substance and an agent for increasing the blood/plasma insulin concentration. The agent may be a carbohydrate, protein, one or more amino acids or a combination of such nutrients

Desirably, the carnitine substance comprises one or more of carnitine, a functional equivalent of carnitine, an active derivative of carnitine or a carnitine analogue. A preferred embodiment may comprise one or more of L-carnitine, a functional equivalent of L-carnitine, an active derivative of L-carnitine or an analogue thereof. In some embodiments the carnitine substance is L-carnitine L-tartrate.

In some embodiments, where the agent for increasing the blood/plasma insulin concentration is a carbohydrate it may be a simple carbohydrate, e.g. a simple sugar. In some embodiments the carbohydrate comprises glucose, but other sugars can be used, for example sucrose or fructose. The agent may be any dietary nutrient that will elevate blood insulin, e.g. a dietary nutrient able to elevate blood/plasma insulin levels to about 50 mU/L or more. The agent may be protein or one or more amino acids or a combination or any two or more of protein, amino acid and carbohydrate.

Compositions of the invention, a carnitine substance for use of the invention, or an agent for increasing the blood/plasma insulin concentration for use of the invention may be provided in the form of a solution which may be an aqueous solution. Alternatively, the compositions, carnitine or agent may be provided in the form of one or more powders.

The inventors have assessed whether 12 weeks of L-carnitine and carbohydrate feeding reduced whole-body fat accumulation compared to carbohydrate feeding alone in young, healthy male volunteers in positive energy balance. Furthermore, the inventors assessed whether muscle carnitine elevation following 12 weeks of L-carnitine and carbohydrate feeding was associated with increased rates of whole-body fatty acid oxidation and energy expenditure during low intensity physical activity, and was accompanied by the maintenance of maximal muscle CPT1 activity in healthy volunteers despite this chronic carbohydrate feeding. To provide further mechanistic insight, specifically regarding muscle genomic adaptation in response to any carnitine mediated increase in muscle fat oxidation and altered metabolic flux, the inventors used pathway focussed, quantitative, real time PCR-based low density arrays to measure changes in mRNA abundance of 187 genes involved in carnitine and fuel metabolism.

The skilled man will appreciate that preferred features of any one embodiment and/or aspect of the invention may be applied to all other embodiments and/or aspects of the invention.

The present invention will be further described in more detail, by way of example only, with reference to the following figures in which:

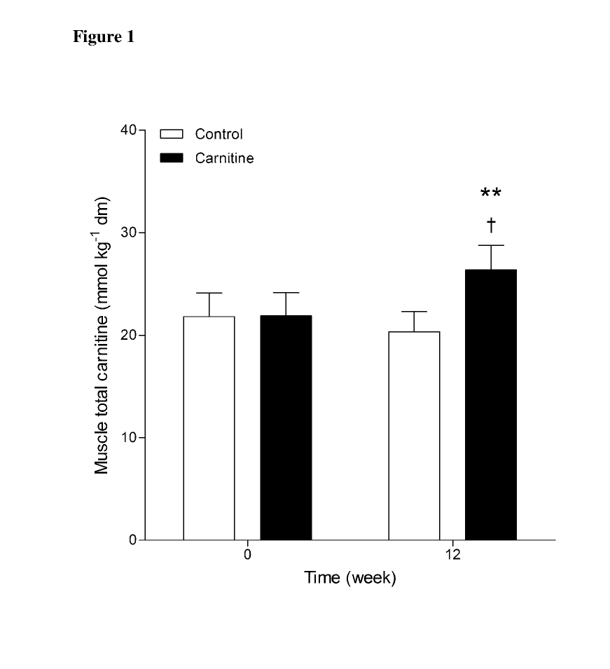

FIG. 1 shows resting skeletal muscle total carnitine content before (0) and 12 weeks after twice daily oral ingestion of either 80 g of carbohydrate (Control; n=6) or 80 g of carbohydrate containing 2 g L-carnitine L-tartrate (Carnitine; n=6). All values are means±standard error of the mean (SEM). **P<0.01, significantly greater than week 0; † P<0.05, significantly greater than Control at 12 weeks.

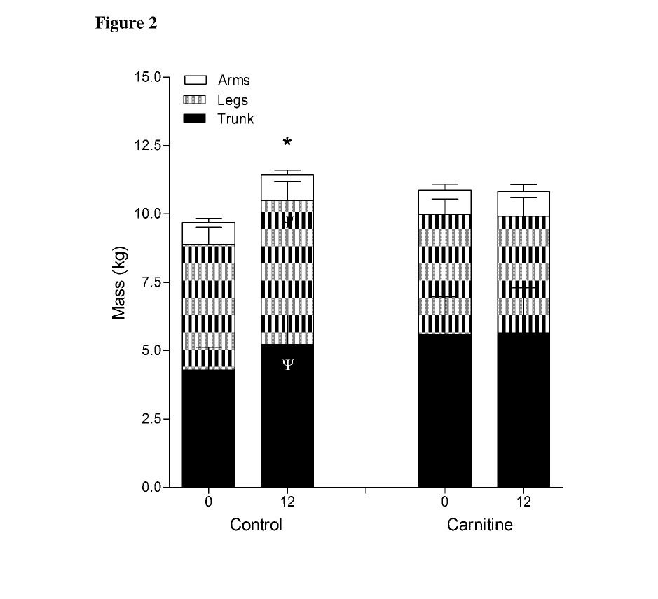

FIG. 2 shows body fat content before (0) and 12 weeks after twice daily oral ingestion of either 80 g of carbohydrate (Control; n=6) or 80 g of carbohydrate containing 2 g L-carnitine L-tartrate (Carnitine; n=6). All values are means±standard error of the mean (SEM). *P<0.05, total body fat mass significantly greater than week 0; ^{Ψ P<}0.05, leg and trunk fat mass significantly greater than week 0.

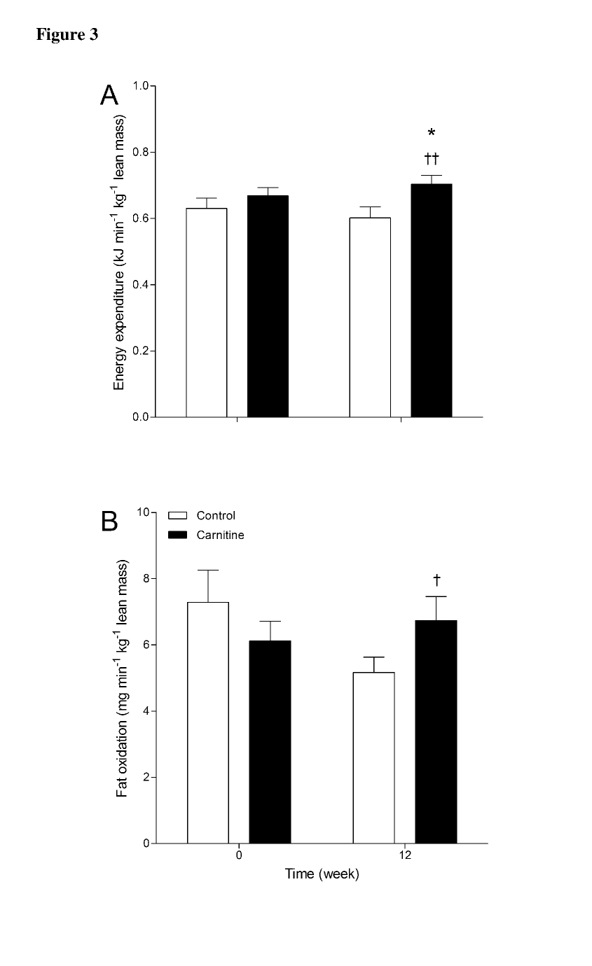

FIG. 3 shows whole body energy expenditure (A) and fat oxidation (B) during 20 min of cycling exercise at 50% VO₂max before (0) and 12 weeks after twice daily oral ingestion of either 80 g of carbohydrate (Control; n=6) or 80 g of carbohydrate containing 2 g L-carnitine L-tartrate (Carnitine; n=6). All values are means±standard error of the mean (SEM). *P<0.05, significantly greater than 0; †† P<0.01, † P<0.05, change over 12 weeks significantly greater than Control.

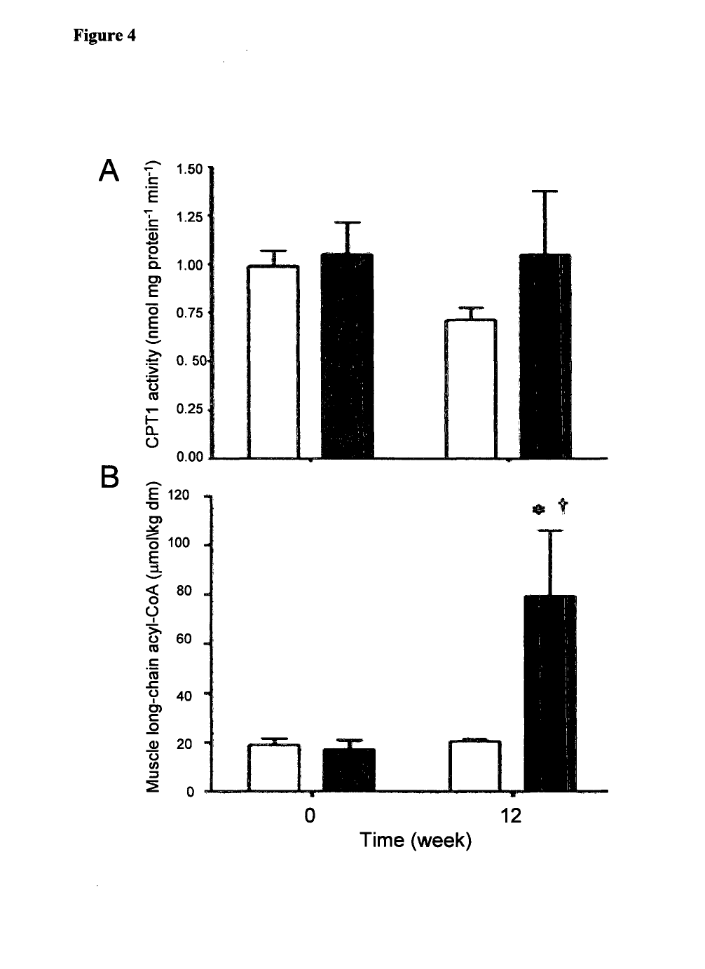

FIG. 4 shows resting skeletal muscle CPT1 maximal activity (A; n=5) and long-chain acyl-CoA content (B; n=6) before (0) and 12 weeks after twice daily oral ingestion of either 80 g of carbohydrate (Control) or 80 g of carbohydrate containing 2 g L-carnitine tartrate (Carnitine). All values are means±standard error of the mean (SEM). *P<0.05, significantly greater than week 0; † P<0.05, significantly different from corresponding Control at 12 weeks.

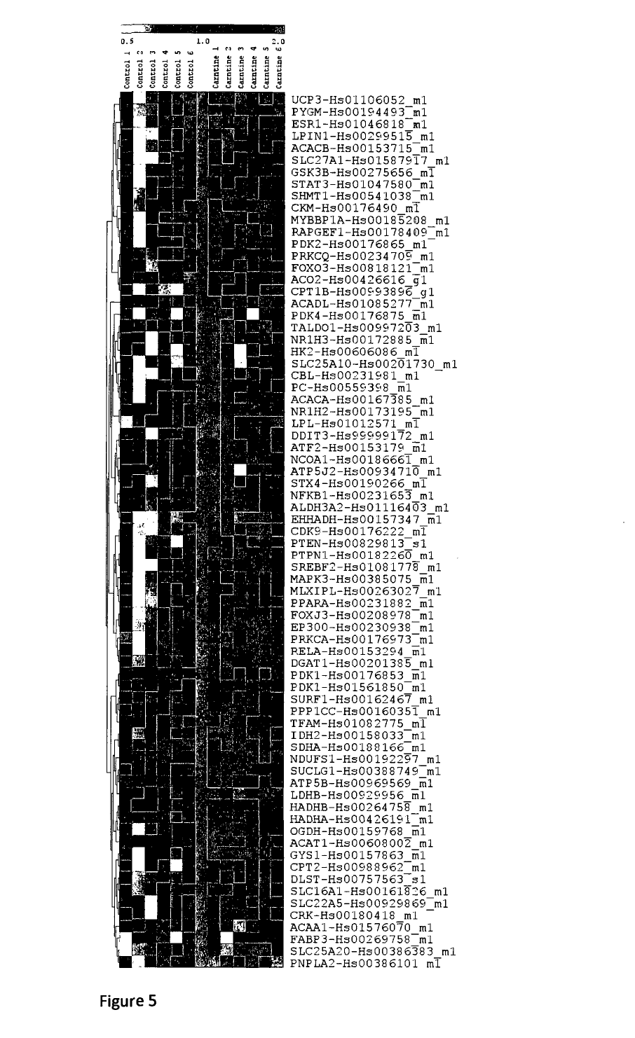

FIG. 5 shows a heat map of skeletal muscle transcripts involved in fuel metabolism pathways after 12 weeks of twice daily oral ingestion of either 80 g of carbohydrate (Control) or 80 g of carbohydrate containing 2 g L-carnitine tartrate (Carnitine). All values are expressed as relative mRNA abundance compared to baseline (week 0) from individual subjects (1-6) in each group.

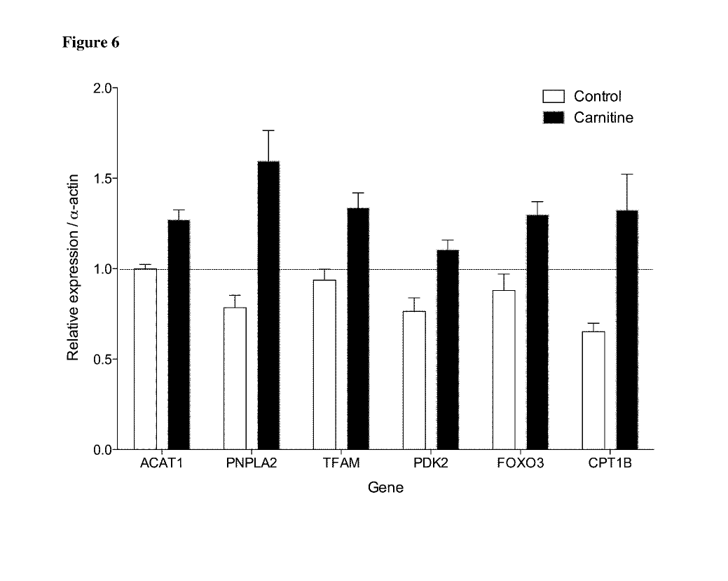

FIG. 6 shows the 6 genes that were the most significantly differentially expressed when comparing Control and Carnitine groups (ACAT, PNPLA2, TFAM, PDK2, FOXO3, CPT1B). Adjusting the false discovery rate to <1%.

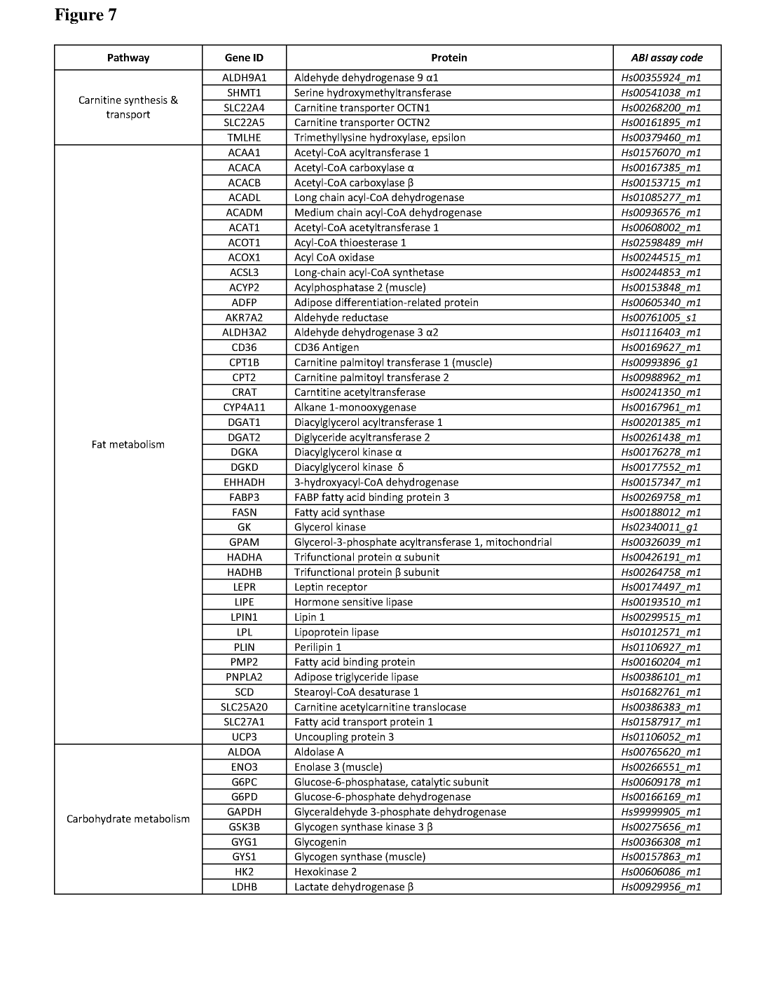

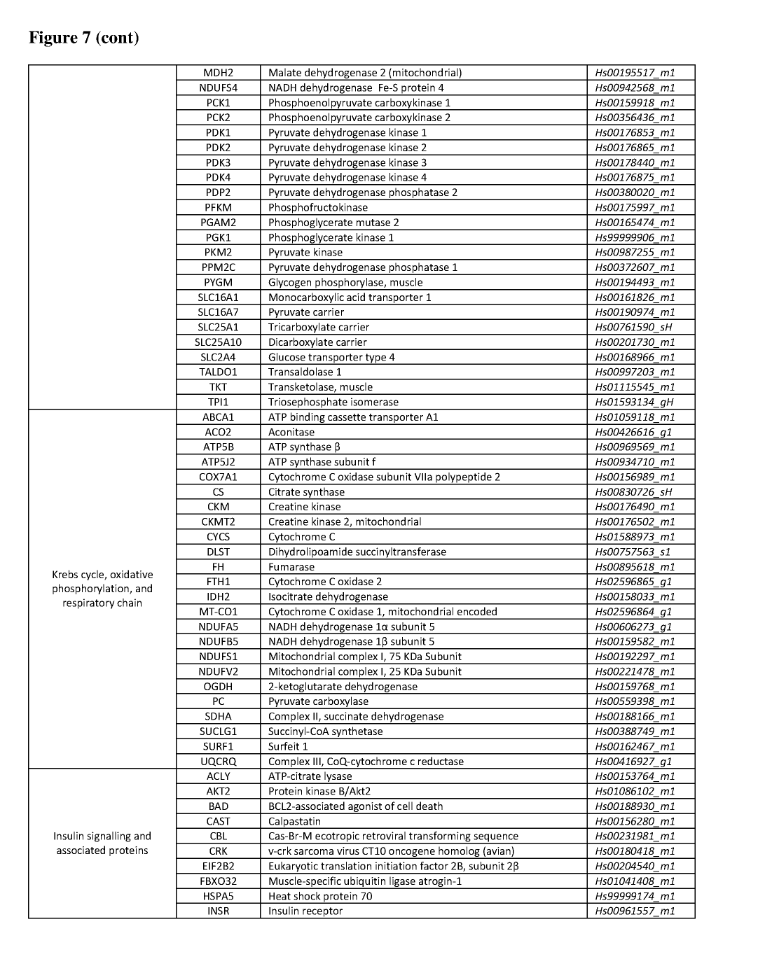

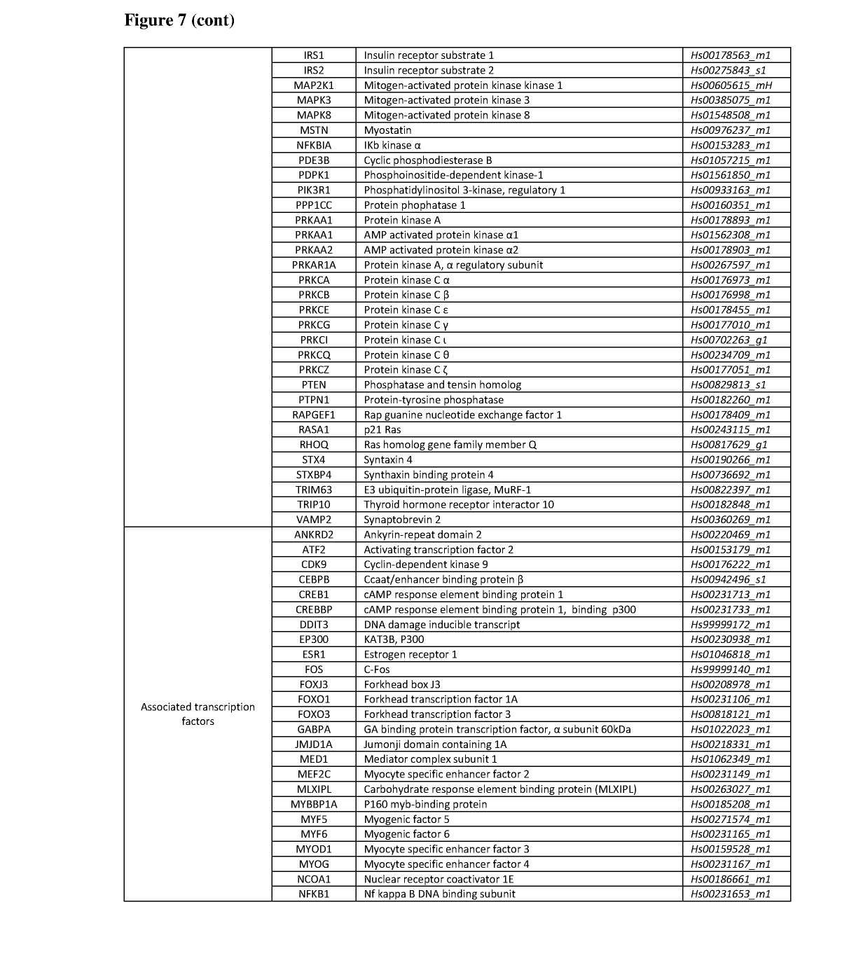

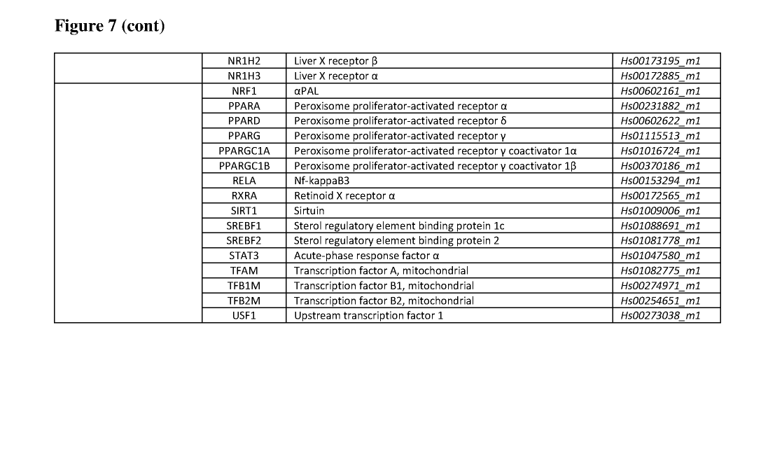

FIG. 7 shows a list of genes encoding proteins involved in pathways of carnitine and fuel metabolism used on low density RT-PCR array microfludic cards (Applied Biosystems Inc., Foster City, Calif., USA) in combination with the ABI PRISM 7900T sequence detection system and SDS 2.1 software (Applied Biosystems Inc., Foster City, Calif., USA). RT-PCR assay codes for each gene are also provided.

### ABBREVIATIONS

The following abbreviations are used throughout: ACC2, acetyl-CoA carboxylase 2; CPT1, carnitine palmitoyltransferase 1; C_{T}, threshold cycle; DEXA, dual energy x-ray absorptiometry; FDR, false discovery rate; KEGG, Kyoto Encyclopedia of Genes and Genomes; PCR, polymerase chain reaction; PDC, pyruvate dehydrogenase complex; PPARa, peroxisome proliferator activated receptor alpha; SAM, significance analysis of microarray; VO₂max, maximal oxygen consumption; PDK2, pyruvate dehydrogenase kinase 2; FOXO3, forkhead box class O transcription factor 3a.

### EXAMPLES

Methods

Ethical Approval

Twelve healthy, non-vegetarian, recreationally active male volunteers participated in the present study, which was approved by the University of Nottingham Medical School Ethics Committee in accordance with the Declaration of Helsinki. Before taking part in the study, all subjects underwent routine medical screening, completed a general health questionnaire, and performed an incremental exercise test to exhaustion on a cycle ergometer (Excalibur, Lode, Groningen, The Netherlands) in order to determine their rate of maximal oxygen consumption (VO₂max), measured using an online gas analysis system (Vmax, SensorMedics, Anaheim, Calif., USA). All gave their written consent to take part and were aware that they were free to withdraw from the experiments at any point. Before the first experimental visit the subjects were allocated in a randomised, double blind manner into two experimental groups as described previously (Wall et al. 2011); Control (age 25.3±2.1 y, body mass index 22.1±0.9 kg m⁻², and VO₂max 3.6±0.3 1 min⁻¹) and Carnitine (age 28.5±2.1 y, body mass index 24.4±0.8 kg m⁻², and VO₂max 4.1±0.1 1 min ⁻¹).

Experimental Protocol

On two occasions separated by 12 weeks, volunteers reported to our laboratory following an overnight fast and having abstained from strenuous exercise and alcohol consumption for the previous 48 h. On arrival at the laboratory subjects were weighed and had their body composition measured by dual energy x-ray absorptiometry (DEXA; Lunar Prodigy, GE Medical Systems, Bucks, U.K.). Volunteers then exercised for 20 min on the cycle ergometer at a workload corresponding to 50% VO₂max, with oxygen consumption and a carbon dioxide production measurements obtained for 3 minute periods at 7 and 17 min. Following the first experimental visit, subjects were instructed to consume a 700 ml orange flavoured beverage twice daily for 24 weeks containing either 80 g of carbohydrate (Vitargo; Swecarb AB, Stockholm, Sweden; Control) or 80 g of carbohydrate in combination with 2 g of L-carnitine L-tartrate (equating to 1.36 g of L-carnitine; Lonza Group Ltd., Basel, Switzerland; Carnitine). Volunteers were asked to ingest the first supplement at breakfast time and the second 4 h later, in order to maximize the time when plasma carnitine concentration was elevated in the presence of increased circulating insulin (Stephens et al. 2007 J Appl Physiol 102, 1065-70).

Sample Collection and Analysis

A single operator (see acknowledgments) analysed all DEXA scans to determine lean soft tissue mass (kg), fat mass (kg), and bone mineral content (kg) of standard body regions. The scans were analyzed for leg, arm, and trunk composition using the standardized regions specified by the manufacturer (enCORE 2005 version 9.1, GE Medical Systems, Bucks, U.K.). Percentage body fat was calculated by dividing fat mass by the sum of fat, lean, and bone mass.

On each experimental visit, a resting muscle sample was obtained from the vastus lateralis using the percutaneous needle biopsy technique (Bergstrom, 1975 Scand J Clin Lab Invest 35, 609-616). Muscle samples were immediately frozen in liquid nitrogen after removal from the limb. One portion of each biopsy sample was freeze dried and stored at −80° C., whilst the remainder was stored ‘wet’ in liquid nitrogen. Freeze dried muscle was dissected free of visible blood and connective tissue, pulverised and used for the determination free-, acetyl-, and long-chain acyl-carnitine content (Cederblad et al. 1990 Anal Biochem 185, 274-278). Total carnitine was calculated as the sum of these carnitine moieties and has been presented in part elsewhere (Wall et al. 2011 J Physiol 589, 963-973). Long-chain acyl-CoA content was determined from the same extract as long-chain acylcarnitine using a modified version of the radioenzymatic method of Cederblad et al (1990) as described previously (Stephens et al. 2006 J Clin Endocrinol Metab 91, 5013-8).

Approximately 20 mg of wet muscle tissue was used to determine maximal CPT1 activity using the forward radioisotope assay (McGarry et al. 1983 Biochem J 214, 21-28). Briefly, muscle was homogenised in 50 mM Tris/HCl buffer (pH 7.5) and immediately used to determine malonyl-CoA (10 μM) sensitive [¹⁴C]palmitoylcarnitine production from 100 μM palmitoyl-CoA, 1 mM L-carnitine, and 0.05 μCi L-[¹⁴C]carnitine, which was normalized to total protein content using the Bradford assay.

In addition, total RNA was extracted from approximately 20 mg of wet muscle tissue by the method of Chomczynski and Sacchi (1987 Anal Biochem 162, 156-159) using Trizol reagent (Invitrogen Ltd, Paisley, UK). Following spectrophotometric quantification, first-strand cDNA was generated from 2 μg of RNA using the SuperScript III cDNA kit (Invitrogen Ltd, Paisley, UK) and stored at −80° C. Thereafter, the relative mRNA abundance of 187 genes from pathways involved in carnitine metabolism, lipid metabolism, carbohydrate metabolism, mitochondrial function (Krebs cycle; oxidative phosphorylation; respiratory chain), insulin signalling, along with associated transcription factors, was determined in duplicate using custom designed low density RT-PCR array microfludic cards (Applied Biosystems Inc., Foster City, Calif., USA) in combination with the ABI PRISM 7900T sequence detection system and SDS 2.1 software (Applied Biosystems Inc., Foster City, Calif., USA). The candidate genes were selected from pathway analysis software (Ingenuity Systems Inc., Redwood City, Calif., USA), Pubmed literature searches, and data obtained from our laboratory. A complete list of details for each gene assay is available in Supplement 1. The threshold cycle C_{T} was automatically given by the SDS software RQ manager, and relative mRNA abundance was calculated using the ΔΔC_{T} method with each subjects' baseline sample (0 week) as their own calibrator and α-actin as the endogenous control. C_{T} values for α-actin did not change across time points (data not shown).

Calculations and Statistical Analysis

Indirect calorimetry calculations were performed according to the non-protein stoichiometric equations given by Frayn (1983 J App Physiol 55, 628-34). Total energy expenditure during exercise was calculated as the sum of energy production from fat and carbohydrate, assuming that the oxidation 1 g of triacylglycerol (862 g mol⁻¹) liberates 39.4 kJ and 1 g of glucose (180 g mol⁻¹) liberates 15.6 kJ, and was normalised to lean body mass (DEXA). A two-way ANOVA (time and treatment effects) was performed to detect differences within and between treatment groups for all measures described with the exception of mRNA abundance (see below). When a significant effect was observed, a Student's t-test was performed to locate individual differences. Statistical significance was declared at P<0.05. Due to the relatively large number of genes measured, Significance of Microarray (SAM) analysis was performed between the relative mRNA abundance values for Control and Carnitine on a two-class unpaired basis with 924 permutations and a false discovery rate of <5% (MeV 4.5, TM4; Saeed et al. 2006 Methods in Enzymology 411, 134-93). Hierarchical cluster analysis using Pearson's correlation was subsequently performed on the genes with a significant differential expression (MeV 4.5, TM4), as well as gene enrichment analysis against the KEGG pathway (DAVID 6.7, NIH; Huang et al. 2009 Nature Protoc 4, 44-57). The original gene list, as opposed to the whole human genome, was used as a background for the enrichment analysis of the significant genes in order to eliminate any bias from the selection of the genes on the original list. All the values presented in text, Tables and Figures represent mean±the standard error of the mean.

Results

### Example 1

Muscle Total Carnitine Content

Resting skeletal muscle total carnitine content at baseline (0 week) was similar in Control and Carnitine (21.8±2.3 vs. 21.9±2.3 mmol kg⁻¹ dm, respectively; FIG. 1). However, whereas 12 weeks of carbohydrate supplementation had no effect on the muscle total carnitine pool in Control (20.3±2.0 mmol kg⁻¹ dm), daily L-carnitine and carbohydrate supplementation for 12 weeks increased muscle total carnitine content in every subject by 21% on average to 26.4±2.4 mmol kg⁻¹ dm (P<0.01), such that the change in total carnitine from baseline was different to Control (4.5±0.9 vs. −1.5±2.3 mmol kg⁻¹ dm in Carnitine and Control, respectively; P<0.05).

### Example 2

Body Composition

Total body mass calculated by DEXA increased in every subject in Control over 12 weeks (75.1±4.2 to 77.0±4.1 kg, respectively; P<0.05), but did not change in Carnitine (76.6±2.3 and 76.7±2.2 kg, respectively. The 1.9±0.7 kg increase in body mass over 12 weeks in Control was entirely accounted for by an increase of 1.8±0.7 kg in body fat mass (P<0.05; FIG. 2), 1.0±0.4 kg of which was due to increased trunk fat mass (P<0.05; FIG. 2), and 0.7±0.2 kg was due to increased leg fat mass (P<0.05; FIG. 2). Whole body, trunk, and leg fat mass increased in every subject in Control. Lean body mass did not change in Control (60.3±2.8 and 60.1±2.6 kg, respectively) or Carnitine over 12 weeks (61.5±1.2 and 61.9±1.4 kg, respectively).

### Example 3

Low Intensity Exercise

The volunteers cycled at similar absolute workloads of 115±13 and 137±12 W in Control and Carnitine, respectively, at 0 and 12 weeks. Whereas energy expenditure during exercise in Control remained relatively constant over 12 weeks (0.63±0.03 vs. 0.60±0.03 kJ min⁻¹ kg⁻¹ lean mass, respectively; FIG. 3A), energy expenditure increased by 6% in Carnitine from a similar baseline value of 0.66±0.02 kJ min⁻¹ kg⁻¹ lean mass to 0.70±0.03 kJ min⁻¹ kg⁻¹ lean mass after 12 weeks (P<0.05; FIG. 3A). The change in energy expenditure from baseline was positive in every subject in Carnitine and markedly different to Control (36.0±12.0 vs. −26.9±12.6 J min⁻¹ kg⁻¹ lean mass in Carnitine and Control, respectively; P<0.01; FIG. 3A), and likely due to the differences in fat oxidation as carbohydrate oxidation during exercise did not change from baseline after 12 weeks of supplementation in Control (22.0±1.6 vs. 25.8±2.4 mg min⁻¹ kg⁻¹ lean mass, respectively) or Carnitine (27.3±1.5 vs. 28.1±1.7 mg min⁻¹ kg⁻¹ lean mass). For example, although fat oxidation during exercise did not differ significantly from baseline after 12 weeks in Control (7.3±1.0 vs. 5.2±0.5 mg min⁻¹ kg⁻¹ lean mass, respectively; FIG. 3B), there was a trend for it to increase in Carnitine (6.1±0.6 vs. 6.7±0.7 mg min⁻¹ kg⁻¹ lean mass, respectively; P=0.09), such that the change in the rate of fat oxidation from baseline was markedly different between Carnitine and Control (0.6±0.3 vs. −2.1±1.2 mg min⁻¹ kg⁻¹ lean mass, respectively; P<0.05 interaction effect; FIG. 3B).

### Example 4

Muscle CPT1 Activity and Long-Chain Acyl-CoA Content

There were no differences in skeletal muscle maximal CPT1 activity before or after 12 weeks in Control or Carnitine (FIG. 4A). Skeletal muscle total long-chain acyl-CoA content under resting fasted conditions did not change after 12 weeks in control, but increased from a similar baseline concentration by approximately 4-fold after 12 weeks in Carnitine (P<0.05), such that it was also 4-fold greater than Control at this corresponding time-point (P<0.05; FIG. 4B).

### Example 5

Gene Expression

Seventy-three genes were differentially expressed (<5% FDR) between Control and Carnitine over 12 weeks, with the expression of all genes being greater in Carnitine (mean fold change 0.92±0.01 vs. 1.33±0.02, respectively; Table 1). The individual responses for each subjects' fold change from baseline (0 week) for the 73 genes are shown on the heatmap in FIG. 5, which also depicts the gene cluster following hierarchical analysis. Gene functional analysis highlighted ‘insulin signalling pathway’ (ACACA, ACACB, CBL, CRK, GSK3B, GYS1, HK2, MAPK3, PPP1G, PTPN1, PYGM, RAPGEF1, SHC1; P=9.3×10⁻¹²), ‘PPAR signalling pathway’ (ACSL3, CPT1B, CPT2, CYP4A11, EHHADH, FABP3, LPL, NR1H3, PPARA; SLC27A1; P=2.9×10⁻¹⁰), and ‘fatty acid metabolism’ (ACAT1, ACSL3, ALDH3A2, CPT1B, CPT2, CYP4A11, EHHADH, HADHA, HADHB; P=7.8×10⁻¹⁰) as the top three enriched pathways from the Kyoto Encyclopedia of Genes and Genomes (KEGG). Adjusting the FDR to <1% in order to provide further insight as to the genes with the most significant differential expression between Control and Carnitine revealed 6 transcripts (ACAT, PNPLA2, TFAM, PDK2, FOXO3, CPT1B; highlighted in Table 1 and FIG. 6).

TABLE 1
Expression of skeletal muscle transcripts encoding proteins involved
in carnitine and fuel metabolism pathways after 12 weeks of twice
daily oral ingestion of either 80 g of carbohydrate (Control) or 80 g
of carbohydrate containing 2 g L-carnitine L-tartrate (Carnitine).

| Pathway | Gene ID | Control | Carnitine |
|---|---|---|---|
| Carnitine synthesis & | SHMT1 | 0.97 ± 0.07 | 1.29 ± 0.08 |
| transport | SLC22A5 | 0.88 ± 0.08 | 1.36 ± 0.17 |
| Fat metabolism | ACAA1 | 0.91 ± 0.03 | 1.27 ± 0.17 |
| | ACACA | 0.95 ± 0.11 | 1.54 ± 0.35 |
| | ACACB | 0.79 ± 0.12 | 1.14 ± 0.14 |
| | ACADL | 1.10 ± 0.06 | 1.36 ± 0.16 |
| | ACAT1 | 1.00 ± 0.02 | 1.27 ± 0.05 |
| | ALDH3A2 | 0.98 ± 0.08 | 1.49 ± 0.24 |
| | CD36 | 0.94 ± 0.11 | 1.22 ± 0.25 |
| | CPT1B | 0.65 ± 0.05 | 1.32 ± 0.20 |
| | CPT2 | 0.81 ± 0.08 | 1.24 ± 0.14 |
| | DGAT1 | 0.94 ± 0.07 | 1.28 ± 0.09 |
| | EHHADH | 0.78 ± 0.11 | 1.62 ± 0.42 |
| | FABP3 | 0.80 ± 0.12 | 1.25 ± 0.25 |
| | HADHA | 0.91 ± 0.07 | 1.27 ± 0.11 |
| | HADHB | 1.03 ± 0.09 | 1.47 ± 0.16 |
| | LPIN1 | 0.81 ± 0.13 | 1.08 ± 0.12 |
| | LPL | 0.89 ± 0.08 | 1.93 ± 0.59 |
| | PNPLA2 | 0.78 ± 0.07 | 1.59 ± 0.17 |
| | SLC25A20 | 0.80 ± 0.10 | 1.23 ± 0.16 |
| | SLC27A1 | 0.76 ± 0.12 | 1.09 ± 0.12 |
| | UCP3 | 0.92 ± 0.12 | 1.39 ± 0.38 |
| Carbohydrate metabolism | GSK3B | 0.92 ± 0.07 | 1.22 ± 0.08 |
| | GYS1 | 0.87 ± 0.08 | 1.13 ± 0.05 |
| | HK2 | 0.85 ± 0.19 | 1.64 ± 0.51 |
| | LDHB | 1.08 ± 0.14 | 1.86 ± 0.46 |
| | PDK1 | 1.03 ± 0.10 | 1.33 ± 0.11 |
| | PDK2 | 0.76 ± 0.08 | 1.11 ± 0.05 |
| | PDK4 | 0.80 ± 0.18 | 1.67 ± 0.59 |
| | PYGM | 0.92 ± 0.09 | 1.22 ± 0.18 |
| | SLC16A1 | 0.86 ± 0.06 | 1.24 ± 0.15 |
| | SLC25A10 | 0.79 ± 0.10 | 1.51 ± 0.50 |
| | TALDO1 | 1.01 ± 0.06 | 1.39 ± 0.29 |
| Krebs cycle, oxidative | ACO2 | 0.81 ± 0.05 | 1.11 ± 0.07 |
| phosphorylation, and | ATP5B | 1.00 ± 0.09 | 1.25 ± 0.12 |
| respiratory chain | ATP5J2 | 1.12 ± 0.15 | 1.56 ± 0.33 |
| | CKM | 0.94 ± 0.07 | 1.16 ± 0.09 |
| | DLST | 0.99 ± 0.15 | 1.38 ± 0.20 |
| | IDH2 | 0.89 ± 0.07 | 1.13 ± 0.11 |
| | NDUFS1 | 1.03 ± 0.05 | 1.30 ± 0.15 |
| | OGDH | 0.91 ± 0.10 | 1.32 ± 0.12 |
| | PC | 0.90 ± 0.16 | 1.59 ± 0.44 |
| | SDHA | 0.99 ± 0.08 | 1.30 ± 0.11 |
| | SUCLG1 | 0.95 ± 0.06 | 1.16 ± 0.09 |
| | SURF1 | 1.08 ± 0.11 | 1.34 ± 0.09 |
| Insulin signalling and | CBL | 0.81 ± 0.12 | 1.52 ± 0.46 |
| associated proteins | CRK | 0.92 ± 0.09 | 1.35 ± 0.12 |
| | MAPK3 | 0.98 ± 0.14 | 1.30 ± 0.13 |
| | PDPK1 | 1.02 ± 0.09 | 1.30 ± 0.05 |
| | PPP1CC | 1.04 ± 0.09 | 1.30 ± 0.06 |
| | PRKCA | 0.83 ± 0.08 | 1.13 ± 0.10 |
| | PRKCQ | 0.97 ± 0.08 | 1.28 ± 0.08 |
| | PTEN | 0.82 ± 0.11 | 1.18 ± 0.17 |
| | PTPN1 | 0.84 ± 0.12 | 1.08 ± 0.08 |
| | RAPGEF1 | 0.84 ± 0.12 | 1.19 ± 0.06 |
| | STX4 | 0.99 ± 0.11 | 1.34 ± 0.22 |
| Associated transcription | ATF2 | 1.06 ± 0.08 | 1.31 ± 0.14 |
| factors | DDIT3 | 1.03 ± 0.13 | 1.38 ± 0.24 |
| | EP300 | 0.98 ± 0.11 | 1.24 ± 0.08 |
| | ESR1 | 1.01 ± 0.12 | 1.24 ± 0.07 |
| | FOXJ3 | 0.94 ± 0.12 | 1.18 ± 0.10 |
| | FOXO3 | 0.88 ± 0.09 | 1.30 ± 0.07 |
| | MLXIPL | 0.95 ± 0.17 | 1.33 ± 0.17 |
| | MYBBP1A | 0.80 ± 0.10 | 1.06 ± 0.05 |
| | NCOA1 | 1.08 ± 0.10 | 1.33 ± 0.12 |
| | NFKB1 | 1.13 ± 0.11 | 1.45 ± 0.21 |
| | NR1H2 | 0.88 ± 0.03 | 1.12 ± 0.11 |
| | NR1H3 | 1.26 ± 0.16 | 2.30 ± 0.96 |
| | PPARA | 0.87 ± 0.13 | 1.31 ± 0.12 |
| | RELA | 0.93 ± 0.11 | 1.28 ± 0.14 |
| | SREBF2 | 0.85 ± 0.13 | 1.17 ± 0.07 |
| | STAT3 | 0.95 ± 0.11 | 1.32 ± 0.10 |
| | TFAM | 0.94 ± 0.06 | 1.34 ± 0.08 |

All values (n = 6) are means ± standard error of the mean (SEM) and expressed as relative mRNA abundance compared to baseline (week 0).

Discussion

The general aim of the present study was to investigate whether increasing muscle carnitine content over a 12 week period during which L-carnitine was consumed in combination with a high carbohydrate beverage could result in the modulation of human energy metabolism. With this in mind, a major finding was that a 20% increase in muscle carnitine content prevented the 1.8 kg increase in body fat mass associated with daily ingestion of a high carbohydrate beverage. Moreover, this maintenance of body mass was associated with a markedly greater whole-body energy expenditure during low intensity physical activity, accounted for by an increase in fat oxidation, and a marked adaptive increase in the expression of gene networks involved in insulin signalling, peroxisome proliferator activated receptor (PPAR) signalling, and fatty acid metabolism. Taken together the present findings suggest that increasing skeletal muscle carnitine content prevented the increased adiposity observed with prolonged supplementation of a high carbohydrate beverage by maintaining the capacity to oxidise fat.

Twelve weeks of twice daily supplementation with a beverage containing 80 g of carbohydrate in Control resulted in a 1.8 kg increase in body mass, which was almost entirely attributable to an increase in trunk and leg fat. Assuming adipose tissue contains 30 MJ kg⁻¹ this equates to a positive energy balance of approximately 640 kJ day⁻¹ and suggests that the volunteers in the Control group were ‘overfed’ carbohydrate. We propose, therefore, that the increase in muscle carnitine content obviated the effect of a long-term increase in daily energy intake from, predominantly, carbohydrate. Specifically, whilst chronic daily carbohydrate overfeeding had no effect on energy expenditure during low intensity exercise in Control, which is in agreement with previous reports, energy expenditure during exercise increased by 0.04 kJ min⁻¹ kg⁻¹ lean mass over 12 weeks in Carnitine such that it was 0.06 kJ min⁻¹ kg⁻¹ lean mass greater than Control. Thus, for the Carnitine group (61 kg lean mass) to maintain whole-body fat mass and balance the 640 kJ day⁻¹ increase in energy intake from carbohydrate overfeeding observed in Control, both groups would have had to perform just under 3 hours of physical activity at equivalent of 50% VO₂max per day, which was certainly feasible for the recreationally active cohort of the present study. The mechanism behind the increase in energy expenditure is not entirely clear, but it is most likely due to the greater change in the rate of fat oxidation observed after 12 weeks in Carnitine compared to Control, in the absence of a change in the rate of carbohydrate oxidation. As the absolute workload during exercise did not change in the present study, and therefore presumably the ATP demand of muscle contraction, it would suggest that mitochondrial ATP synthesis was less efficient with greater fat utilisation. Indeed, unlike glycolysis, the catabolism of fatty acids to acetyl-CoA produces FADH as an electron donor, which has a lower P/O ratio than NADH, and a well characterised adaptation to excess fatty acid oxidation is an increase in mitochondrial uncoupling. Furthermore, an increased rate of fat oxidation during low intensity exercise (50% VO₂max) has been linked with increased energy expenditure and reduced mitochondrial efficiency during state 3 respiration (i.e. oxygen consumption for a given amount of ADP), and more efficient mitochondrial ATP production and increased P/O ratio during exercise has been associated with increased carbohydrate oxidation during low intensity exercise (50% VO₂max). Muscle free carnitine content was not measured after 20 min of exercise in the present study, but in our previous study a 20% increase in skeletal muscle total carnitine content following 24 weeks of daily L-carnitine and carbohydrate feeding resulted in a striking 80% greater availability of muscle free carnitine following 30 minutes of low intensity exercise compared to Control, which was associated with a 30% reduction in PDC activation and 55% reduction in muscle glycogen utilisation. The present study shows that these previous findings were likely due to a relative increase in the rate of fatty acid oxidation during exercise with a dietary mediated increase in the muscle total carnitine store. That energy expenditure and fat oxidation increase at rest in the Carnitine group seems highly likely given there was a 4-fold increase in resting muscle long-chain acyl-CoA content.

To provide mechanistic insight of the interaction between chronically altered metabolic flux and muscle gene expression (independent of changes in mitochondrial content), we used a pathway focussed, quantitative, real time PCR-based low density array to determine coordinated expression of genes involved in the regulation of muscle fuel metabolism. The relative mRNA abundance of 73 of the 187 genes measured was increased in the Carnitine group compared Control after 12 weeks, with gene functional analysis highlighting ‘insulin signalling’, ‘PPAR signalling’ and ‘fatty acid metabolism’ as the 3 most enriched functional pathways. The finding that gene networks within the insulin signalling pathway were upregulated with a prevention of adiposity, particularly in the abdominal region, suggests that insulin sensitivity may have been increased in Carnitine. Indeed, the gene for pyruvate dehydrogenase kinase 2 (PDK2), a key negative regulator of PDC activation that is upregulated in response to a prolonged increase in fatty acid flux, was highlighted in the <1% FDR gene-set as greater in Carnitine compared to Control. Together with greater mRNA abundance of forkhead box class O transcription factor 3a (FOXO3), a transcriptional controller of PDK expression that was also highlighted in the <1% FDR gene-set and clustered with PDK2, adds further mechanistic weight to an increase in muscle carnitine content resulting in a switch in fuel use towards fat oxidation. The mechanism(s) by which increased fatty acid flux can improve insulin sensitivity was not investigated in the present study, but it would likely involve manipulation of intramyocellular lipid and the associated metabolites, which are known to impair insulin signalling, particularly as muscle long-chain acyl-CoA was greater in Carnitine in the present study. Of note, the transcript for adipose triglyceride lipase (ATGL or PNPLA2), a key controlling enzyme in intramyocellular lipid hydrolysis, also had a 1% FDR and clustered tightly with the transcripts for carnitine acylcarnitine translocase (SLC25A20) and fatty acid binding protein (FABP3), which are both involved in mitochondrial fat translocation.

The upregulation of ‘PPAR signalling’ and ‘fatty acid metabolism’ pathways clearly support our conclusion that the prolonged modulation of energy metabolism associated with an increase in muscle carnitine content was due to an increase in fatty acid flux. This is particularly remarkable as high carbohydrate ingestion has been shown to blunt increases in fat oxidative genes in response to stimuli that promote a switch to fat use, and the abundance of numerous fat oxidative genes was decreased in Control of the present study. In particular, the transcripts for acetyl-CoA acetyltransferase 1 (ACAT1), ATGL, and CPT1 were highlighted in the <1% FDR gene-set, with the latter 2 genes down-regulated in every Control volunteer. Furthermore, common to both functional pathways are genes regulated by PPARα, which is a nuclear receptor protein involved in lipid sensing and the modulation of the expression of many key genes involved in fatty acid transport and metabolism. Both saturated and unsaturated long-chain acyl-CoAs have been demonstrated to be very high-affinity ligands for PPARα, and accumulation of intracellular fatty acyl-metabolites has been suggested to activate PPARα in viv, which is consistent with 4-fold greater long-chain acyl-CoA content after 12 weeks in Carnitine of the present study. PPARα is also the main transcriptional regulator of CPT1 in skeletal muscle, the mRNA expression of which was 2-fold greater in Carnitine than Control at 12 weeks. Thus, we propose that increasing muscle carnitine content resulted in an increase in muscle CPT1 flux and long-chain acyl-CoA content, which in turn signaled a molecular adaptation to maintain greater fat oxidation. Consistent with the overall premise of the present study, PPARα activation in a hamster model of obesity increased mitochondrial β-oxidation 1.6-fold in skeletal muscle without affecting glucose utilisation, resulting in increased energy expenditure and 17% body mass loss.

In conclusion, this is the first study to demonstrate that increasing skeletal muscle carnitine content in healthy humans can modulate energy metabolism over a prolonged period, as reflected by a prevention of adiposity, an increase in energy expenditure during low intensity exercise, and a robust increase in the expression of metabolic genes regulating muscle fuel selection in response to 12 weeks of carbohydrate overfeeding In line with the role of carnitine in the translocation of long-chain acyl-groups via CPT1, these important findings are most likely due to an increase in the rate of fat oxidation compared to Control at rest and during low intensity exercise. These findings have clear health implications, particularly as there is accumulating evidence that there may be a primary, possibly genetically, determined component of fat oxidation, which may result in a low rate of fat oxidation predisposing subjects to the development of obesity and insulin resistance.

To summarise the results and discussion herein demonstrate that twelve weeks of daily L-carnitine and carbohydrate feeding increases skeletal muscle total carnitine content and prevents body mass accrual associated with CHO feeding alone. Here we determined the influence of prolonged L-carnitine and CHO feeding on energy metabolism, whole-body fat mass, and genes involved in muscle fuel metabolism. Twelve healthy male volunteers (age 27±2y, BMI 23.3±0.7 kg m⁻²) exercised at 50% VO₂max for 20 min before and after 12 weeks twice daily feeding of 80 g carbohydrate (Control) or 1.36 g L-carnitine +80 g carbohydrate (carnitine). Muscle total carnitine did not change over 12 weeks in Control, but increased in carnitine by 20% (P<0.05). Energy expenditure during exercise remained constant over 12 weeks in Control (0.63±0.03 vs. 0.60±0.03 kJ min⁻¹ kg⁻¹ lean mass), but increased in Carnitine from 0.66±0.02 to 0.70±0.03 kJ min⁻¹ kg⁻¹ after 12 weeks (P<0.05). As a result, whole-body fat mass increased over 12 weeks in Control by 18% (1.8±0.6 kg; P<0.05), but did not change in Carnitine. Seventy-three of 187 muscle fuel metabolism genes were upregulated in Carnitine vs. Control after 12 weeks, with ‘insulin signalling’, ‘PPAR signalling’ and ‘fatty acid metabolism’ as the three most enriched pathways in gene functional analysis. Thus, increasing muscle TC in healthy humans can modulate energy expenditure and fat metabolism over a prolonged period, and could be beneficial to health as perturbed muscle fat metabolism predisposes to the development of insulin resistance.

1. A carnitine substance for use in the reduction of whole-body fat mass accumulation, wherein the carnitine substance is administered or used as a dietary supplement together with an agent for increasing the blood/plasma insulin concentration.

2. An agent for use in the reduction of whole-body fat mass accumulation, wherein the agent is administered or used as a dietary supplement together with administration of, or dietary supplementation with a carnitine substance, and wherein the agent is suitable for increasing the blood/plasma insulin concentration.

3. A carnitine substance for use in the treatment of insulin resistance, fat oxidation disorders or obesity, wherein the carnitine substance is administered or used as a dietary supplement together with an agent for increasing the blood/plasma insulin concentration.

4. An agent for use in the treatment of insulin resistance, fat oxidation disorders or obesity, wherein the agent is administered or used as a dietary supplement together with administration of, or dietary supplementation with a carnitine substance, and wherein the agent is suitable for increasing the blood/plasma insulin concentration.

5. A carnitine substance for use in increasing energy expenditure and fat metabolism in a subject, wherein the carnitine substance is administered or used as a dietary supplement together with an agent for increasing the blood/plasma insulin concentration.

6. An agent for use in increasing energy expenditure and fat metabolism in a subject, wherein the agent is administered or used as a dietary supplement together with administration of or dietary supplementation with a carnitine substance, and wherein the agent is suitable for increasing the blood/plasma insulin concentration.

7. A composition comprising a carnitine substance and an agent for increasing the blood/plasma insulin concentration for use in the reduction of whole-body fat mass accumulation, the treatment of insulin resistance, fat oxidation disorders or obesity, or increasing energy expenditure and fat metabolism in a subject.

8. A kit comprising a carnitine substance and an agent for increasing the blood/plasma insulin concentration, and instructions for use in the reduction of whole-body fat mass accumulation, the treatment of insulin resistance, fat oxidation disorders or obesity, or increasing energy expenditure and fat metabolism in a subject.

9. A method for reducing whole-body fat mass accumulation comprising administering an agent for increasing the blood/plasma insulin concentration and a carnitine substance to an individual or supplementing the diet of an individual with an agent for increasing the blood/plasma insulin concentration and a carnitine substance.

10. A method for treating insulin resistance, fat oxidation disorders or obesity comprising administering an agent for increasing the blood/plasma insulin concentration and a carnitine substance to an individual or supplementing the diet of an individual with an agent for increasing the blood/plasma insulin concentration and a carnitine substance.

11. A method for increasing energy expenditure and fat metabolism in a subject, comprising administering an agent for increasing the blood/plasma insulin concentration and a carnitine substance to an individual or supplementing the diet of an individual with an agent for increasing the blood/plasma insulin concentration and a carnitine substance.

12. A carnitine substance for use of any of claim 1, 3 or 5, an agent for use of any of claims 2, 4 or 6, or a kit of claim 8, wherein the carnitine substance and agent for increasing the blood/plasma insulin concentration are administered simultaneously, separately, or sequentially.

13. A method of any of claims 9 to 11 wherein the carnitine substance and agent for increasing the blood/plasma insulin concentration are administered simultaneously, separately, or sequentially.

14. The invention of any preceding claim wherein the carnitine substance is administered or fed at a dose of about 0.5 to 4 g per day, preferably at a dose of about 1 to 3 g per day, e.g. 5 to 2.5 g per day.

15. The invention of any preceding claim wherein the carnitine substance is administered or fed over one or more doses per day, e.g. one dose per day, two doses per day, or three or more doses per day.

16. The invention of any preceding claim wherein the carnitine substance is administered or fed at about 2.72 g per day, e.g. about 1.36 g twice per day.

17. The invention of any preceding claim wherein the agent for increasing the blood/plasma insulin concentration is administered or fed at a total dose of about 20 g to about 200 g per day, preferably at a total dose of about 50 g to 150 g per day, e.g about 100 g to 125 g.

18. The invention of any preceding claim wherein the agent is administered or fed in one or more doses per day, e.g. one dose per day, two doses per day, or three or more doses per day.

19. The invention of any preceding claim wherein the agent is administered or fed at about 160 g per day, e.g. about 80 g twice per day.

20. The invention of any preceding claim wherein the dose of the agent is adequate to elevate blood/plasma insulin to about 50 mU/L or more.

21. An invention of any preceding claim wherein the administration of, or dietary supplementation with an agent for increasing the blood/plasma insulin concentration and a carnitine substance results in an upregulation of the expression of any one or more of the genes in Table 1.

22. An invention of claim 21 wherein the expression of one or more genes involved in insulin signalling, PPAR signalling or fatty acid metabolism is upregulated by the administration or dietary supplementation with a carnitine substance and an agent for increasing the blood/plasma insulin concentration.

23. An invention of claim 22 wherein the expression of one or more of ACAT1, PNPLA2, PDK2, FOXO3, TFAM or CPT1 is upregulated by the administration of, or dietary supplementation with a carnitine substance and an agent for increasing the blood/plasma insulin concentration.

24. An invention of any preceding claim wherein the carnitine substance is L-carnitine.

25. An invention of any preceding claim wherein the agent for increasing the blood/plasma insulin concentration is a carbohydrate, protein, one or more amino acid, or a combination of two or more such dietary nutrients.

26. An invention of any preceding claim wherein the agent for increasing the blood/plasma insulin concentration is capable of elevating the blood/plasma insulin concentration to about 50 mU/L or more.
